(19) United States
(12) Patent Application Publication
(10) Pub. No.: US 2025/0002859 A1
(43) Pub. Date: Jan. 2, 2025

(54) METHOD FOR PRODUCING CULTURED MEAT BY UTILIZING FBS SUBSTITUTE DERIVED FROM BUTCHERY BY-PRODUCTS

(71) Applicant: CHUNG-ANG UNIVERSITY INDUSTRY-ACADEMIC COOPERATION FOUNDATION, Seoul (KR)

(72) Inventors: Sun Jin HUR, Gyeonggi-do (KR); Da Young LEE, Gyeonggi-do (KR); Seung Yun LEE, Gangwon-do (KR); Jae Won JEONG, Gyeonggi-do (KR); Jae Hyeon KIM, Busan (KR); Hyun Woo KIM, Seoul (KR)

(21) Appl. No.: 18/712,889

(22) PCT Filed: Oct. 28, 2022
(86) PCT No.: PCT/KR2022/016758

§ 371 Date: May 23, 2024

(30) Foreign Application Priority Data
Nov. 25, 2021 (KR) 10-2021-0164624
Oct. 28, 2022 (KR) 10-2022-0141590

(51) Int. Cl. C12N 5/077; A23L 13/00; B01D 61/14

(52) U.S. Cl. CPC C12N 5/0659; A23L 13/00; B01D 61/145; B01D 61/147

(57) ABSTRACT

The present invention relates to a method for producing cultured meat by utilizing a fetal bovine serum (FBS) substitute derived from butchery by-products and, more specifically, to: a method for preparing a medium additive (an FBS substitute) for culturing cultured meat prepared by the method; a medium composition for culturing cultured meat comprising the additive; and a method for preparing cultured meat by using same. By using the medium additive (the FBS substitute) for culturing cultured meat prepared by the unique method of the present invention, the traditional fetal bovine serum (FBS), which has a high unit cost and the ethical issue of having to be collected from live calves, is completely substituted or jointly used, and thus the amount of fetal bovine serum (FBS) use can be reduced and the product efficiency of the cultured meat can be enhanced. Further, by increasing the utilization of slaughter by-products which are inevitably generated during butchering of livestock for producing meat, the present invention provides the advantages of increasing the value of the butchery by-products and increasing economic advantages such as reduced disposal costs and increased incomes.

### TECHNICAL FIELD

The present invention relates to a method for producing cultured meat using a fetal bovine serum (FBS) substitute derived from butchery by-products. More specifically, it relates to a method of preparing a medium additive for culturing cultured meat (FBS substitute) from slaughter by-products, a medium additive for culturing cultured meat prepared by the method, a medium composition for culturing cultured meat comprising the additive, and a method for producing cultured meat using the composition.

The present invention claims priority based on Korean Patent Application No. 10-2021-0164624 filed on Nov. 25, 2021, and No. 10-2022-0141590 filed on Oct. 28, 2022, and all contents disclosed in the specifications and drawings of these applications are incorporated herein by reference.

### BACKGROUND ART

According to a report by the Food and Agriculture Organization (FAO) of the United Nations, the world population is projected to increase to 9.5 billion by 2050. However, due to global warming and other climate anomalies, crop yields are expected to decrease while the demand for grain for feed will increase, leading to higher production costs and reduced production areas. As a result, animal-derived food products are predicted to become expensive.

Although animal-derived foods are costly in terms of energy input and production, they have been directly contributing to human nutrition by providing high-quality proteins and micronutrients essential for normal human growth and health. Especially, essential amino acids, which are not synthesized or synthesized in very small amounts in the body and must be ingested through food, indicate that the demand for animal-derived foods will reach 550 million tons by 2050, twice the current amount. This shows the limitations of traditional livestock production methods in providing the necessary protein for essential amino acids.

As a solution to future meat shortages, ‘cultured meat’ has been gaining attention recently as an alternative to traditional livestock farming. Cultured meat is edible meat obtained by culturing cells taken from living animals using cell engineering technology, a field of cellular agriculture that produces meat without farming livestock. In Korean, it is referred to as [symbol] [symbol] or [symbol] and in English, it is known as in vitro meat, implying meat grown in a test tube, artificial meat, indicating meat synthesized using stem cells by humans and not natural, clean meat, signifying meat produced in clean, non-traditional farming facilities, and lab-grown meat, as some prototypes are made in laboratories.

Cultured meat primarily uses muscle satellite cells, and other cells such as embryonic stem cells, induced pluripotent stem cells, and adipose-derived adult stem cells have sufficient differentiation ability but have not been successfully differentiated into muscle tissue. In contrast, muscle satellite cells, which play a regenerative role in skeletal muscle injury, differentiate only into muscle tissue, making them most suitable for cultured meat production.

The most important substance in the cell culture medium used for the proliferation and differentiation of cultured meat is fetal bovine serum (FBS). FBS can be obtained from the blood fraction collected from the fetus in the womb of a slaughtered pregnant cow. Compared to general serum, FBS contains fewer antibodies and more growth factors, making it effective for cell culture. It is obtained from the fetus before birth and contains minimal antibodies, reducing the likelihood of immune reactions in various cells. Besides, FBS used in biotechnology research, such as vaccine and pharmaceutical development, is expected to increase in demand. However, to obtain FBS, which is essential for cell culture, pregnant cows must be slaughtered and their fetuses used, leading to unstable supply and high cost.

To solve these disadvantages of FBS, various studies have been conducted using serum-free (FBS free) media, but the serum-free media have the disadvantage of significantly slower cell division and growth rates. Other technologies for FBS substitution in South Korea include the development of serum-free biomaterials based on marine organisms as a substitute for FBS, as in South Korea Registered Patent No. 10-1950245, but the actual feasibility of widespread use of these is unknown.

Therefore, there is a need for the development of alternative substances that can solve the aforementioned problems associated with FBS and are effective for cell culture.

### DISCLOSURE

### Technical Problem

The present invention aims to solve the problems of the conventional technology as described above. The inventors have been researching a method to produce a qualified fetal bovine serum (FBS) substitute, which can be used for cell culture, by utilizing butchery by-products from animals such as cattle, pigs, or chickens that are otherwise discarded. During this research, it was discovered that using the FBS substitute (a medium additive for culturing cultured meat) prepared by the unique method of the present invention can completely replace or be used in conjunction with fetal bovine serum (FBS), which is conventionally expensive and ethically controversial as it is collected from living calves. This discovery also revealed that the FBS substitute can improve the production efficiency of cultured meat, thus completing this invention.

Therefore, the present invention is directed to providing a method for preparing a medium additive for culturing cultured meat (FBS substitute) from butchery by-products, comprising:
(i) a step of obtaining serum or plasma from blood separated from animals other than humans as a by-product of butchering:
(ii) a step of bleaching the obtained serum or plasma from the step (i); and
(iii) a step of filtering the bleached serum or plasma obtained from the step (ii).

The present invention is also directed to providing the medium additive for culturing cultured meat (FBS substitute) prepared by the manufacturing method of the present invention.

Additionally, the present invention is directed to providing a medium composition for culturing cultured meat comprising the medium additive (FBS substitute) of the present invention.

Moreover, the present invention is directed to providing a method for producing cultured meat, comprising a step of proliferating muscle satellite cells using the medium composition for culturing cultured meat of the present invention.

However, the technical problems that the present invention seeks to solve are not limited to those mentioned above, and other problems not mentioned will become clearly understood to those skilled in the art from the description below.

### Technical Solution

To achieve the objectives as mentioned above, the present invention provides a method for preparing a medium additive for culturing cultured meat from butchery by-products, comprising:
(i) a step of obtaining serum or plasma from blood separated from animals other than humans as a by-product of butchering:
(ii) a step of bleaching the obtained serum or plasma from the step (i); and
(iii) a step of filtering the bleached serum or plasma obtained from the step (ii).

In one embodiment of the present invention, the animal is one or more selected from the group consisting of cattle, chickens, and pigs, but is not limited thereto.

In one embodiment of the present invention, the cattle may be 30 to 48 months of age, the chickens may be 2 to 8 weeks of age, and the pigs may be 180 days of age, but are not limited thereto.

In one embodiment of the present invention, the bleaching in the step (ii) may be performed by mixing kaolin with the serum or plasma obtained in the step (i), but is not limited thereto.

In one embodiment of the present invention, the bleaching in the step (ii) may be performed at a temperature of 50 to 70° C., but is not limited thereto.

In one embodiment of the present invention, the filtering process in the step (iii) may be characterized by filtering through pores sized 0.05 to 0.3 μm in diameter, but is not limited thereto.

In one embodiment of the present invention, the filtering process in the step (iii) may further include a pre-filtering process through pores sized 2 μm to 25 μm in diameter, but is not limited thereto.

In one embodiment of the present invention, the filtering in the step (iii) may be characterized by using a membrane filter, but is not limited thereto.

In one embodiment of the present invention, the filtering may additionally include methods selected from the group consisting of filter paper, syringe filter, and combinations thereof, in addition to filtration by a membrane filter, but is not limited thereto.

In one embodiment of the present invention, the method for preparing a medium additive for culturing cultured meat (FBS substitute) from butchery by-products of the present invention may further include a sterilization process after the filtering process in the step (iii), but is not limited thereto.

In one embodiment of the present invention, the sterilization may be performed at a temperature of 50 to 70° C., but is not limited thereto.

To achieve another objective of the present invention, it provides the medium additive for culturing cultured meat (FBS substitute) prepared by the manufacturing method of the present invention (method for preparing a medium additive for culturing cultured meat from butchery by-products).

In one embodiment of the present invention, the medium additive for culturing cultured meat of the present invention may be characterized as a medium additive for the proliferation of muscle satellite cells, but is not limited thereto.

In one embodiment of the present invention, the medium additive for culturing cultured meat of the present invention may be characterized as substituting for or being used in conjunction with fetal bovine serum (FBS), but is not limited thereto.

To achieve yet another objective of the present invention, it provides a medium composition for culturing cultured meat comprising the medium additive for culturing cultured meat (FBS substitute) of the present invention.

In one embodiment of the present invention, the medium composition for culturing cultured meat of the present invention may be characterized as being for the proliferation of muscle satellite cells, but is not limited thereto.

In one embodiment of the present invention, the medium composition for culturing cultured meat of the present invention may comprise, relative to 100 weight parts of serum-free medium, 1 to 20 weight parts of the medium additive for culturing cultured meat (FBS substitute) and 0.5 to 1.5 weight parts of antibiotics/antimicrobials, but is not limited thereto.

In one embodiment of the present invention, the serum-free medium may be at least one selected from the group consisting of Ham's 10 medium, Ham's 12 medium, DMEM, MEM, MCDB-201 medium, and RPMI 1640 medium, but is not limited thereto.

In one embodiment of the present invention, the antibiotics/antimicrobials may include at least one type of antibiotic selected from penicillin and streptomycin and at least one type of antifungal agent selected from amphotericin-B, clotrimazole, miconazole, ketoconazole, and nystatin, but is not limited thereto.

In one embodiment of the present invention, the medium composition for culturing cultured meat of the present invention may additionally include fetal bovine serum (FBS), but is not limited thereto. In such an embodiment, the medium composition for culturing cultured meat of the present invention may comprise, relative to 100 weight parts of serum-free medium, 10 to 20 weight parts of the medium additive for culturing cultured meat (FBS substitute), 0 to 20 weight parts of fetal bovine serum (FBS), and 0.5 to 1.5 weight parts of antibiotics/antimicrobials, but is not limited thereto.

To achieve another objective of the present invention, it provides a method for preparing cultured meat, comprising a step of proliferating muscle satellite cells using the medium composition for culturing cultured meat of the present invention.

### Advantageous Effects

Using the medium additive for culturing cultured meat (FBS substitute) prepared by the unique method of this invention, it is possible to completely replace or reduce the usage of fetal bovine serum (FBS), which is conventionally expensive and poses ethical issues as it is harvested from living calves, while also improving the production efficiency of cultured meat. Additionally, this invention increases the utility and value of butchery by-products, which are an inevitable outcome of livestock butchering for meat production, leading to economic benefits such as cost savings in waste disposal and increased income, as well as significant environmental protection effects.

Specifically, to address the problems associated with the use of fetal bovine serum (FBS), the inventors of this invention proposed utilizing blood obtained as a by-product of livestock butchering for meat production as an alternative to conventional FBS. By using the FBS substitute (medium additive) produced by the unique method of this invention, utilizing blood obtained as a by-product of livestock butchering, it is possible to resolve the supply instability and high-cost issues of conventional FBS products. Additionally, utilizing these otherwise discarded by-products reduces unnecessary waste disposal costs and generates revenue, while also protecting the environment. These effects ultimately provide a foundation for stably supplying cost-effective additives that can promote growth not only in cell cultures for cultured meat production but also in various other cell cultures.

### DESCRIPTION OF THE DRAWINGS

FIG. 1 is a schematic diagram briefly illustrating the process of producing a fetal bovine serum (FBS) substitute using livestock blood in the method of the present invention.

FIG. 2 shows the process of obtaining blood from butchered livestock (chicken) after simulating a typical animal butchering situation in the laboratory using CO₂, and then separating and obtaining serum or plasma from the blood.

FIG. 3 is a diagram illustrating the bleaching process of serum or plasma (serum/plasma).

FIG. 4 illustrates the series of filtering and sterilization processes performed to remove impurities from the serum or plasma (serum/plasma) solution after bleaching.

FIG. 5 is a diagram showing the series of processes to isolate satellite cells from the muscle tissue of animals (livestock).

FIG. 6 shows the results of cytotoxicity tests on bovine cells using the FBS substitute (produced by the method of Example 1 of each bovine plasma (BP), bovine serum (BS), chicken plasma (CP), and chicken serum (CS)) of the present invention, showing no significant cell death-inducing cytotoxicity.

FIG. 7 shows the results of cytotoxicity tests on chicken satellite cells using the FBS substitute (produced by the method of Example 1 of each bovine plasma (BP), bovine serum (BS), chicken plasma (CP), and chicken serum (CS)) of the present invention, indicating no significant cell death-inducing cytotoxicity.

FIG. 8 is a diagram showing the growth and proliferation of myoblasts cultured with partial application of the FBS substitute (produced by the method of Example 1 of each bovine plasma (BP), bovine serum (BS), chicken plasma (CP), and chicken serum (CS)) in the cell culture medium based on bovine cells (The cell growth and proliferation medium was basically Ham's F-10 medium with 20% (v/v) FBS and 1% (v/v) P/S/A added (CTL, control), and the experiment was conducted with 8 treatment groups replacing 25% (v/v) (5% (v/v) of the total medium composition) or 50% (v/v) (10% (v/v) of the total medium composition) of the total FBS amount with each of the FBS substitutes of the present invention).

FIG. 9 is a diagram showing the degree of differentiation of myoblasts cultured with partial application of the FBS substitute (produced by the method of Example 1 of each bovine plasma (BP), bovine serum (BS), chicken plasma (CP), and chicken serum (CS)) in the cell culture medium based on bovine cells (The cell growth and proliferation medium was basically Ham's F-10 medium with 20% (v/v) FBS and 1% (v/v) P/S/A added (CTL, control), and the experiment was conducted with 8 treatment groups replacing 25% (v/v) (5% (v/v) of the total medium composition) or 50% (v/v) (10% (v/v) of the total medium composition) of the total FBS amount with each of the FBS substitutes of the present invention).

FIG. 10 is a diagram showing the growth and proliferation of myoblasts cultured with partial application of the FBS substitute (produced by the method of Example 1 of each bovine plasma (BP), bovine serum (BS), chicken plasma (CP), and chicken serum (CS)) in the cell culture medium based on chicken cells (The cell growth and proliferation medium was basically Ham's F-10 medium with 20% (v/v) FBS and 1% (v/v) P/S/A added (CTL, control), and the experiment was conducted with 8 treatment groups replacing 25% (v/v) (5% (v/v) of the total medium composition) or 50% (v/v) (10% (v/v) of the total medium composition) of the total FBS amount with each of the FBS substitutes of the present invention).

FIG. 11 is a diagram showing the degree of differentiation of myoblasts cultured with partial application of the FBS substitute (produced by the method of Example 1 of each bovine plasma (BP), bovine serum (BS), chicken plasma (CP), and chicken serum (CS)) in the cell culture medium based on chicken cells (The cell growth and proliferation medium was basically Ham's F-10 medium with 20% (v/v) FBS and 1% (v/v) P/S/A added (CTL, control), and the experiment was conducted with 8 treatment groups replacing 25% (v/v) (5% (v/v) of the total medium composition) or 50% (v/v) (10% (v/v) of the total medium composition) of the total FBS amount with each of the FBS substitutes of the present invention).

Based on bovine cells, FIG. 12 is a graph showing the growth and proliferation of myoblasts cultured in cell culture medium where conventional FBS was completely replaced with the FBS substitute (produced by the method of Example 1 of each bovine serum (BS), porcine serum (PS), and chicken serum (CS)) of the present invention, with each added at concentrations of 20% (v/v), 30% (v/v), or 40% (v/v) within the total cell culture medium (The cell growth and proliferation medium was basically Ham's F-10 medium with 20% (v/v) FBS and 1% (v/v) P/S/A added (CTL, control)).

Based on porcine cells, FIG. 13 is a graph showing the growth and proliferation of myoblasts cultured in cell culture medium where conventional FBS was completely replaced with the FBS substitute (produced by the method of Example 1 of each bovine serum (BS), porcine serum (PS), and chicken serum (CS)) of the present invention, with each added at concentrations of 20% (v/v), 30% (v/v), or 40% (v/v) within the total cell culture medium (The cell growth and proliferation medium was basically Ham's F-10 medium with 20% (v/v) FBS and 1% (v/v) P/S/A added (CTL, control)).

Based on chicken cells, FIG. 14 is a graph showing the growth and proliferation of myoblasts cultured in cell culture medium where conventional FBS was completely replaced with the FBS substitute (produced by the method of Example 1 of each bovine serum (BS), porcine serum (PS), and chicken serum (CS)) of the present invention, with each added at concentrations of 20% (v/v), 30% (v/v), or 40% (v/v) within the total cell culture medium (The cell growth and proliferation medium was basically Ham's F-10 medium with 20% (v/v) FBS and 1% (v/v) P/S/A added (CTL, control)).

### Best Modes

The present invention relates to a method of producing a fetal bovine serum (FBS) substitute intended to replace the expensive and essential FBS for cell growth, utilizing butchery by-products generated during the butchering of livestock. Traditional FBS is produced from the blood collected from stillborn calves within slaughtered mother cows. This production method not only struggles to meet the increasing demand for FBS as cultured meat research intensifies but also involves animal ethical issues. Therefore, the inventors of the present invention focused on creating an effective FBS substitute for cultured meat growth using blood obtained during the butchering of general livestock, rather than from fetuses within the mother body. The inventors devised a method to acquire blood in the form of serum and plasma during the butchering of general livestock and convert it into a form of an FBS substitute. Furthermore, satellite cells necessary for cultured meat production were obtained from the muscles of different livestock species, and the effect of the FBS substitute of the present invention was verified by applying it to cell cultures, comparing it with traditional FBS. In this process, species-specific effects of FBS substitutes derived from cattle, pigs, and chickens were confirmed, and it was also established that plasma, as well as serum, could be used. Additionally, it was ascertained that the FBS substitute of the present invention is effective in forming and growing (proliferating) myoblasts from satellite cells for cultured meat production.

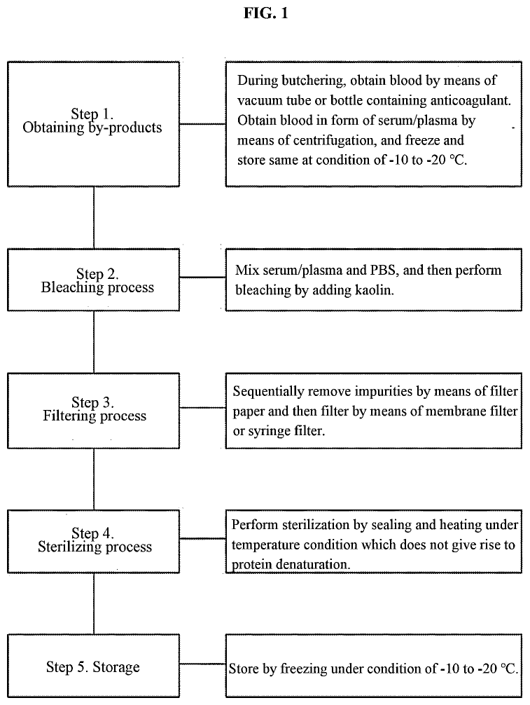

Specifically, the entire process for producing the FBS substitute of the present invention involves obtaining by-products (obtaining serum or plasma from blood), a bleaching process, a filtering process, and a sterilization process. The produced FBS substitute was applied to the cultivation of satellite cells derived from bovine, porcine, and chicken to verify its effectiveness in terms of myoblast growth rate, cell toxicity, and cell differentiation. This process is illustrated in FIG. 1, and its detailed procedures are described in the examples of this specification.

Accordingly, the present invention provides a method for preparing a medium additive for culturing cultured meat (FBS substitute) from butchery by-products, comprising:
(i) a step of obtaining serum or plasma from blood separated from animals other than humans as a by-product of butchering:
(ii) a step of bleaching the obtained serum or plasma from the step (i); and
(iii) a step of filtering the bleached serum or plasma obtained from the step (ii).

In one embodiment of the present invention, the method for preparing the medium additive for culturing cultured meat from butchery by-products is understood to be a method of obtaining an FBS substitute from animals other than humans, comprising the steps (i) to (iii), which will be apparent to those skilled in the art.

In one embodiment of the present invention, the animals may be any mammals excluding humans, and in a preferred embodiment, they may be livestock. In a specific embodiment, the livestock is not limited but may be one or more selected from the group consisting of cattle, pigs, and chickens.

In the step (i), the blood (serum and plasma) is obtained during the slaughtering of the respective animals (livestock), and the slaughtering age of each animal is well known in the art.

In a specific embodiment of the present invention, if the slaughtered animal is cattle, preferably the cattle may be 30 to 48 months of age, more preferably 33 to 46 months of age.

If the slaughtered animal is chicken, preferably the chicken may be 2 to 8 weeks of age, more preferably 2 to 6 weeks of age.

If the slaughtered animal is pig, preferably the pig may be 180 days of age, as the slaughtering age for pigs in South Korea is fixed at 180 days.

In one embodiment of the present invention, preferably in the step (i), the blood (whole blood) separated from animals other than humans as a by-product of butchering may be treated with an anticoagulant. The type of anticoagulant is not particularly limited as long as it is known in the art, and may include one or more selected from the group consisting of ethylenediaminetetraacetic acid (EDTA), ethyleneglycoltetraacetic acid (EGTA), citrate, citrate salt diethylenetriamine pentaacetic acid (DTPA), 1,2-diaminocyclohexane tetraacetic acid (DCTA), heparin, warfarin, and oxalate.

In the present invention, in the step (i), serum or plasma is separated and obtained from the blood (whole blood) separated from animals other than humans as a by-product of butchering. The method of separating serum or plasma from whole blood is known in the art, and the method is not particularly limited in this invention. For example, methods using centrifugation, serum or plasma separating gels, and commercial products for this purpose are known in the art.

In one embodiment of the present invention, the bleaching in the step (ii) may be characterized by mixing kaolin with the serum or plasma obtained in the step (i).

In one embodiment of the present invention, the bleaching in the step (ii) may preferably be performed at a temperature of 50 to 70° C., more preferably at 55 to 65° C. The conditions such as time and stirring in the bleaching step (ii) may be appropriately selected and modified by a practitioner depending on the amount of blood or plasma or the desired bleaching efficiency.

In one embodiment of the present invention, preferably the filtering process in the step (iii) is characterized by filtering through pores sized 0.05 to 0.3 μm in diameter. Not limited thereto, for example, the size of the pores may be 0.05 μm, 0.1 μm, 0.15 μm, 0.2 μm, 0.25 μm, or 0.3 μm, and this invention encompasses all ranges defined by two selected values as boundaries (upper and lower limit). In this case, the filtering process may additionally include a pre-filtering process through pores sized 2 μm to 25 μm in diameter, but is not limited thereto.

The filtering in the present invention is not particularly limited as long as it is a filtering means known in the art. In a preferred embodiment of the present invention, the filtering may be characterized as using a membrane filter, and additional filtering may be performed using methods selected from the group consisting of filter paper, syringe filter, and combinations thereof.

In one embodiment of the present invention, the method for preparing the medium additive for culturing cultured meat (FBS substitute) from butchery by-products may further include a sterilization process after the filtering process in the step (iii), but is not limited thereto.

In a specific embodiment of the present invention, the sterilization may use any blood, serum, or plasma sterilization method known in the art, and the sterilization method is not particularly limited in this invention. Not limited thereto, for example, low-temperature sterilization, radiation irradiation, and other sterilization methods that do not destroy the active ingredients in the FBS substitute (medium additive for culturing cultured meat) of the present invention may be used. In one embodiment of the present invention, the sterilization may be performed at a temperature of 50 to 70° C., and the conditions such as time may be appropriately selected and modified by a practitioner depending on the amount of blood or plasma or the desired sterilization efficiency.

Additionally, the present invention provides a medium additive for culturing cultured meat (FBS substitute), prepared by the manufacturing method of the present invention (method for preparing a medium additive for culturing cultured meat from butchery by-products).

In one embodiment of the present invention, the medium additive for culturing cultured meat of the present invention may be characterized as a medium additive for the proliferation of muscle satellite cells.

In one embodiment of the present invention, the medium additive for culturing cultured meat of the present invention may be characterized as substituting for or being used in conjunction with fetal bovine serum (FBS). Specifically, the medium additive for culturing cultured meat of the present invention may be provided as a substitute for fetal bovine serum (FBS) or as a formulation prepared by additionally adding the medium additive for culturing cultured meat of the present invention to FBS.

In the case of being used in conjunction with fetal bovine serum (FBS), the medium additive for culturing cultured meat of the present invention may replace 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the amount (weight, volume, etc.) of FBS conventionally added to cell culture medium. Here, the % symbol may mean ‘% (v/v)’, and it is also understood as volume part (volume ratio).

Furthermore, the present invention provides a medium composition for culturing cultured meat comprising the medium additive for culturing cultured meat (FBS substitute) of the present invention.

In one embodiment of the present invention, the medium composition for culturing cultured meat of the present invention may be for the proliferation of muscle satellite cells.

In one embodiment of the present invention, the medium composition for culturing cultured meat of the present invention may comprise, relative to 100 weight parts of serum-free medium, 1 to 20 weight parts of the medium additive for culturing cultured meat (FBS substitute) of the present invention, and 0.5 to 1.5 weight parts of antibiotics/antimicrobials.

In one embodiment of the present invention, the medium composition for culturing cultured meat of the present invention may comprise, relative to 100 volume parts of serum-free medium, 1 to 20 volume parts of the medium additive for culturing cultured meat (FBS substitute) of the present invention, and 0.5 to 1.5 volume parts of antibiotics/antimicrobials.

In one embodiment of the present invention, the medium composition for culturing cultured meat of the present invention may be used without including fetal bovine serum (FBS), with the medium additive for culturing cultured meat of the present invention (FBS substitute) fully substituting FBS.

In such a preferred specific embodiment of the present invention, if the medium additive for culturing cultured meat (FBS substitute) is prepared from cattle, the medium composition may include 20 to 40 weight parts (volume parts) (more preferably 20 to 30 weight parts (volume parts)) of the medium additive for culturing cultured meat (FBS substitute) relative to 100 weight parts (volume parts) of serum-free medium. Furthermore, if the medium additive for culturing cultured meat (FBS substitute) is prepared from cattle, it may be preferable to use it for culturing muscle satellite cells derived from cattle or chicken.

If the medium additive for culturing cultured meat (FBS substitute) is prepared from chicken, the medium composition may include 20 to 40 weight parts (volume parts) (more preferably 30 to 40 weight parts (volume parts)) of the medium additive for culturing cultured meat (FBS substitute) relative to 100 weight parts (volume parts) of serum-free medium. Furthermore, if the medium additive for culturing cultured meat (FBS substitute) is prepared from chicken, it may be preferable to use it for culturing muscle satellite cells derived from pigs or chicken.

If the medium additive for culturing cultured meat (FBS substitute) is prepared from pigs, the medium composition may include 20 to 40 weight parts (volume parts) of the medium additive for culturing cultured meat (FBS substitute) relative to 100 weight parts (volume parts) of serum-free medium. Furthermore, if the medium additive for culturing cultured meat (FBS substitute) is prepared from pigs, it may be preferable to use it for culturing muscle satellite cells derived from cattle, pigs, or chicken.

In one embodiment of the present invention, the serum-free medium is not particularly limited as long as it is known in the art for use in cell culture, and may include at least one selected from the group consisting of Ham's (F) 10 medium, Ham's (F) 12 medium, Dulbecco Modified Eagle Medium (DMEM), Minimum Essential Medium (MEM), MCDB-201 medium, and RPMI 1640 medium.

In one embodiment of the present invention, the antibiotics/antimicrobials are not particularly limited as long as they are known in the art for use in cell culture and may include at least one type of antibiotic selected from penicillin and streptomycin, and at least one type of antifungal agent selected from amphotericin-B, clotrimazole, miconazole, ketoconazole, and nystatin, but are not limited thereto.

In one embodiment of the present invention, the medium composition for culturing cultured meat of the present invention may additionally include fetal bovine Serum (FBS), but is not limited thereto. That is, in one embodiment of the present invention, the medium composition for culturing cultured meat of the present invention may be used in a form where the medium additive for culturing cultured meat (FBS substitute) of the present invention is used in conjunction with fetal bovine serum (FBS).

In such an embodiment, the medium composition for culturing cultured meat of the present invention may comprise, relative to 100 weight parts (volume parts) of serum-free medium, 10 to 20 weight parts (volume parts) of the medium additive for culturing cultured meat (FBS substitute) of the present invention, 0 to 20 weight parts (volume parts) of fetal bovine serum (FBS), and 0.5 to 1.5 weight parts (volume parts) of antibiotics/antimicrobials, but is not limited thereto.

Furthermore, the present invention provides a method for preparing cultured meat, comprising a step of proliferating (culturing) muscle satellite cells using the medium composition for culturing cultured meat of the present invention. In a specific embodiment of the present invention, it provides a method for preparing cultured meat, comprising a step of culturing muscle satellite cells in a culture container containing the medium composition for culturing cultured meat of the present invention. In a preferred embodiment of the present invention, the muscle satellite cells may be cultured in a Matrigel-coated culture container (e.g., plate, dish, etc.).

In the present invention, the muscle satellite cells are not particularly limited in terms of the species of the animal from which they are obtained and the age of the animal, and can be obtained according to methods known in the art. In one embodiment of the present invention, the muscle satellite cells may be obtained during butchering in the step (i) of the method for preparing the medium additive for culturing cultured meat from butchery by-products of the present invention.

Hereinafter, preferable examples are presented to aid in the understanding of the present invention. However, these examples are provided merely for easier understanding of the present invention, and the content of the present invention is not limited by these examples.

### Example 1: Obtaining Fetal Bovine Serum (FBS) Substitute from Butchery By-Products

(Step 1) Obtaining Blood from Animal Butchery by-Products, Followed by Obtaining Serum and Plasma.

Blood was obtained during the butchering of livestock. Specifically, cattle blood was obtained while butchering a 45-month-old cow, chicken blood was obtained from about 6-week-old laying hens during butchering, and pig blood was obtained by butchering a 180-day-old gilt.

In cases where heart blood collection was possible, blood was obtained using tubes treated with EDTA (anticoagulant) or coagulant gel. In cases where heart blood collection was not possible, bottles containing a solution dissolved with Na-EDTA were used.

The obtained blood was centrifuged at 3,000 rpm for 10-20 minutes to obtain the supernatant (serum collection).

Blood treated with anticoagulants was centrifuged at 3,000 rpm for 10-20 minutes to obtain the supernatant (plasma collection). The obtained blood in the form of serum/plasma (serum or plasma) was stored frozen at −10 to −20° C.

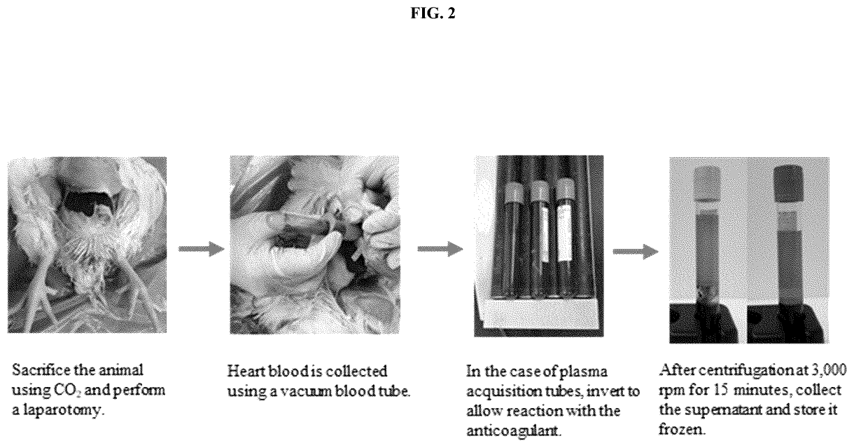

FIG. 2 briefly shows the processes of the step 1 (i.e., collecting blood from butchered animals and obtaining serum or plasma from it).

### (Step 2) Bleaching of Serum/Plasma.

Serum/plasma (50 ml tube) was mixed with 1×PBS (phosphate buffer saline) in a 1:5-10 ratio and then homogenized using a sonicator. Subsequently, 3-5% (v/v) kaolin was added to the mixture and reacted for 30 minutes while shaking in a water bath at 55 to 65° C.

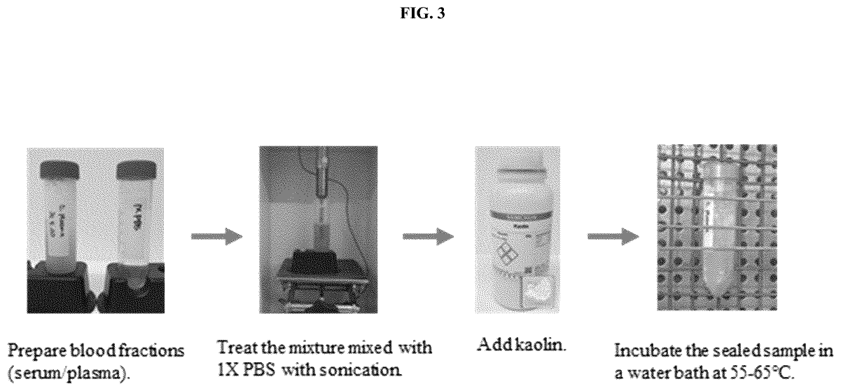

FIG. 3 briefly shows the processes of the step 2.

### (Step 3) Filtering and Sterilization.

520 For removing kaolin and impurities, pre-filtration was performed using filter paper of various sizes (pore size diameter minimum 2 μm to 25 μm). Additional filtration could be performed using a 0.2 μm syringe filter, and the final filtration process was carried out using a 0.1 μm membrane filter, followed by aliquoting into 15/50 ml tubes.

The aliquoted tubes were sealed with Parafilm and reacted for 30 minutes to 1 hour while shaking in a 65° C. water bath for sterilization.

The produced FBS substitute was stored frozen at −10 to −20° C. in tube form.

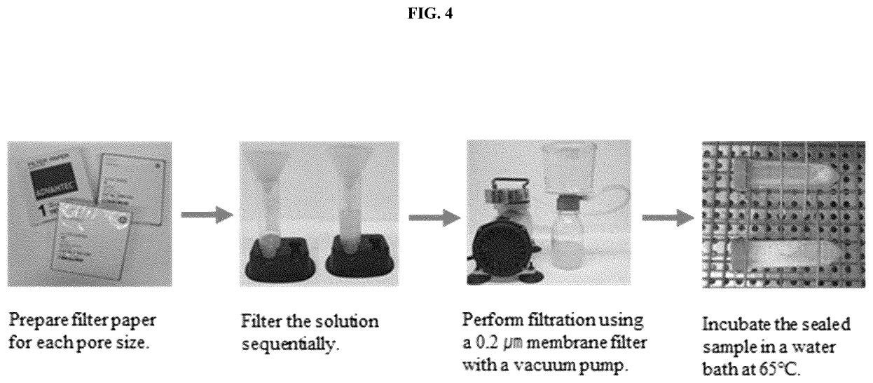

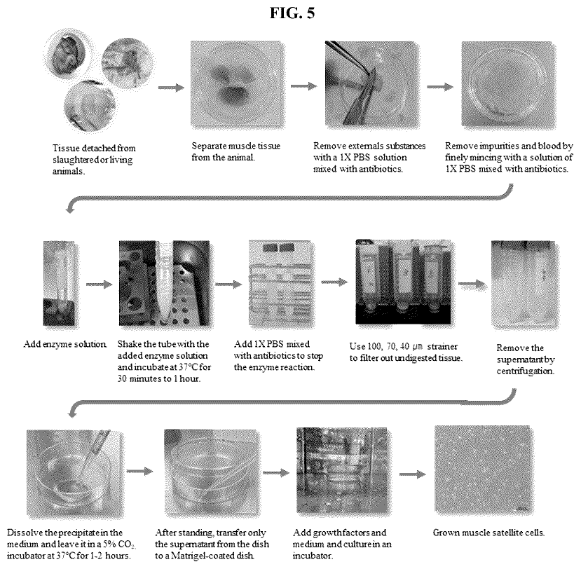

FIG. 4 briefly shows the processes of the step 3.

### Example 2. Separation of Satellite Cells from Animal Muscles

As a step to obtain satellite cells used for cultured meat production, muscle tissues from animals were collected, minced in a 1X PBS solution mixed with antibiotics (1-3% (v/v) penicillin/streptomycin) to remove impurities like blood, fat, and connective tissues. The tissues were then transferred to a tube and digested with an enzyme mixture containing collagenase D (11088858001, Sigma Aldrich, USA) and dipase II (4942074800, Roche, Swiss) at 37° C. for 30 minutes to 1 hour. After the reaction, 1X PBS solution was added to stop the enzyme reaction, un-digested tissues were filtered out, and cells were collected using strainers of 100 μm, 70 μm, 40 μm sizes in sequence. The filtered cell solution was centrifuged to remove the supernatant and collect the cell pellet. The pellet was resuspended in medium (Ham's F-10 (11550043, Gibco, USA)) and left in the incubator for 1-2 hours. After standing, only the supernatant was transferred to a Matrigel-coated dish and cultured by adding growth factors (bFGF, 354060, Corning, USA) and medium. The cell culture medium used was based on Ham's F-10 (11550043, Gibco, USA) with 20% (v/v) FBS (35-015-CV, Corning, USA) and 1% (v/v) P/S/A (03-033-1B, Biological Industries, Israel) added. FIG. 5 briefly shows the processes of separating satellite cells from animal muscle tissues.

### Example 3. Production of Cultured Meat Using the Fetal Bovine Serum (FBS) Substitute of the Present Invention

### 3-1. Verification of Cytotoxicity of the FBS Substitute of the Present Invention

To verify the cytotoxicity of the FBS substitute produced in Example 1, an MTT assay analysis method was used. Thiazolyl Blue Tetrazolium Bromide (sigma M 2128) was used for preparing the MTT reagent. For analysis, each satellite cell obtained from Example 2 was placed in a Matrigel-coated 96 well plate at 1×10⁴ cells and cultured with FBS substitute media prepared in different concentrations.

The experiment was conducted with 8 treatment groups including CTL (control) using a general media composition [Ham's F-10+20% (v/v) FBS+1% (v/v) P/S/A] and each of bovine plasma (BP), bovine serum (BS), chicken plasma (CP), and chicken serum (CS) produced by the method of Example 1, replacing 25% (v/v) (5% of the total media composition) or 50% (v/v) (10% of the total media composition) of the total FBS amount.

After 2-3 days when the cells grew sufficiently, the supernatant was removed. MTT reagent prepared at 5 mg/ml (in 1×PBS) was diluted in media (Ham's F-10) at a 1:9 ratio and added. The plate was wrapped in foil and reacted in a 37° C. 5% CO₂ incubator for 4 hours. Afterwards, the added supernatant was removed, DMSO was added, and reacted again for 10-15 minutes in dark conditions before the absorbance at 540 nm was measured using an ELISA reader.

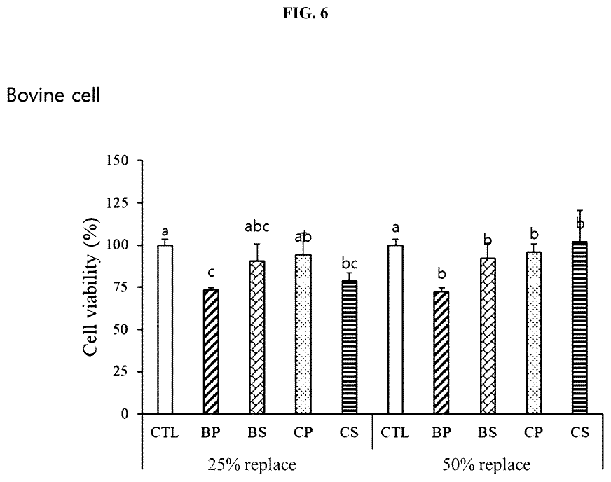

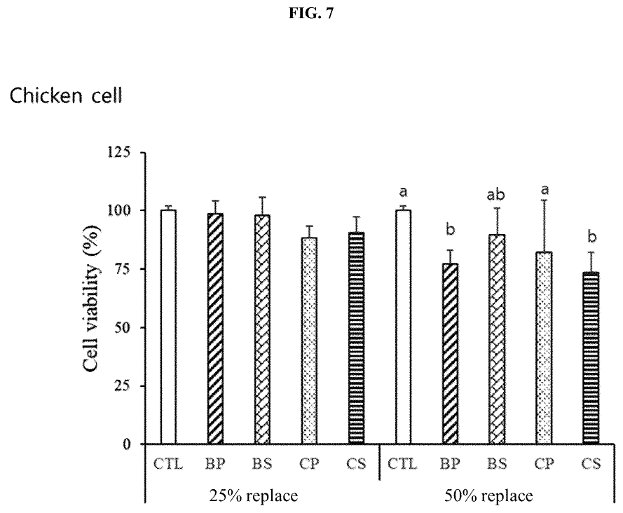

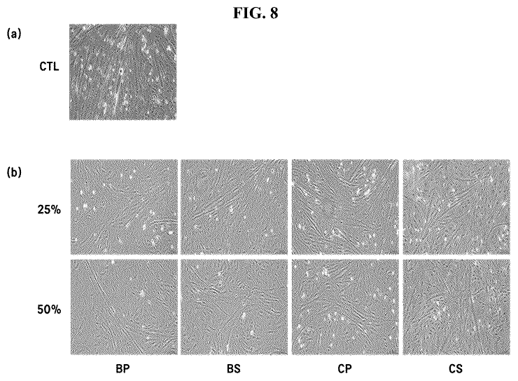

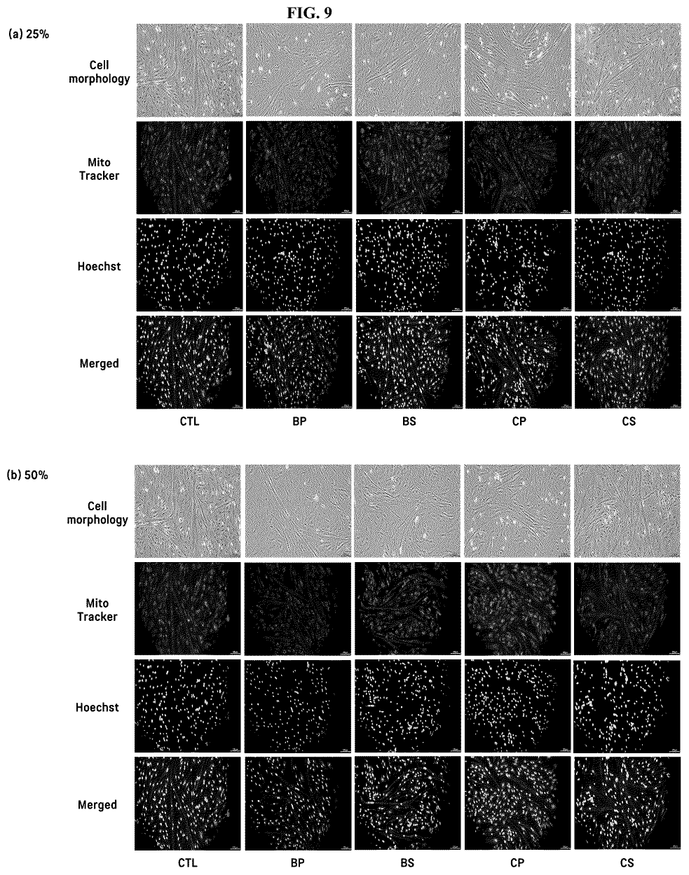

In all treatment groups, the cell toxicity results from using the FBS substitute of the present invention, produced by the method of Example 1 consisting of bovine plasma (BP), bovine serum (BS), chicken plasma (CP), and chicken serum (CS), are as depicted in FIG. 6 and FIG. 7. Since most treatment groups showed similar or slightly higher cell growth compared to CTL, it was determined that the FBS substitute produced by the method of Example 1 can be used for cell culture.

### 3-2. Verification of the Promoting Effect on Myoblast Growth Using the FBS Substitute of the Present Invention Partial Use of the FBS Substitute of the Present Invention as a Substitute for Existing FBS in Growth (Proliferation) Media

After each cell (bovine cell and chicken cell) grew in Example 3-1, the media was replaced to induce differentiation, and the degree of differentiation according to cell growth was confirmed.

The cell growth and proliferation media basically used Ham's F-10 media with 20% (v/v) FBS and 1% (v/v) P/S/A added (CTL, control), and the experiment was conducted with 8 treatment groups replacing 25% (v/v) (5% of the total media composition) or 50% (v/v) (10% of the total media composition) of the total FBS amount with each of bovine plasma (BP), bovine serum (BS), chicken plasma (CP), and chicken serum (CS) produced by the method of Example 1, and the cells were cultured for 3 to 7 days according to cell growth level for measurement.

The cell differentiation media was prepared by adding 2% (v/v) Horse serum and 1% (v/v) P/S/A to DMEM media, and after culturing for 3-5 days according to cell differentiation, it was measured.

The results of cell culture showing cell growth, proliferation, and differentiation degree are shown in FIGS. 8 and 9. As can be seen in FIGS. 8 and 9, myoblasts cultured based on bovine cells showed the most proliferation in the CS treatment group compared to the control group (CTL). When comparing plasma (BP and CP) with serum (BS and CS), more cell proliferation was confirmed in serum for both cattle and chicken blood, and more cell proliferation occurred in the treatment groups using chicken blood compared to cattle blood. Among them, the cell proliferation rate of BP was the lowest, and the proliferation levels of other treatment groups were similar. Therefore, when the proliferated cells were differentiated, although the cell density was relatively low, it was confirmed that the cells were differentiated in a similar form to the control group in all substitute treatment groups.

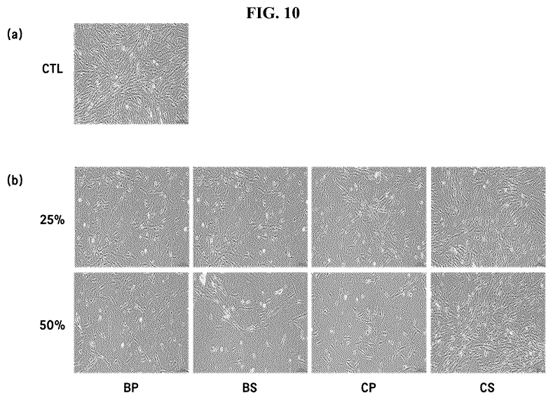

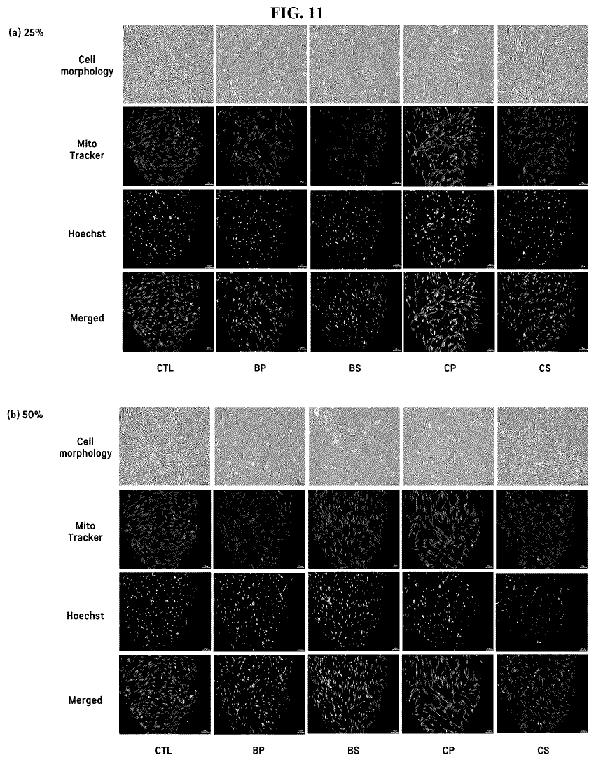

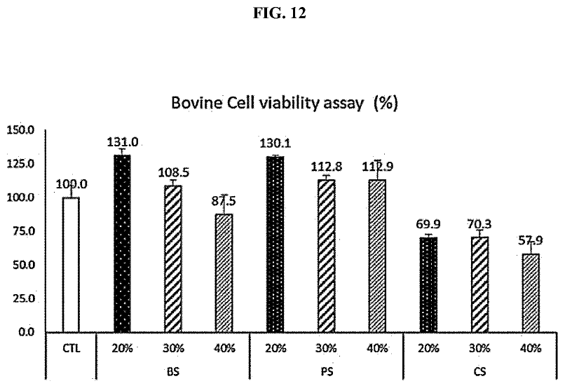

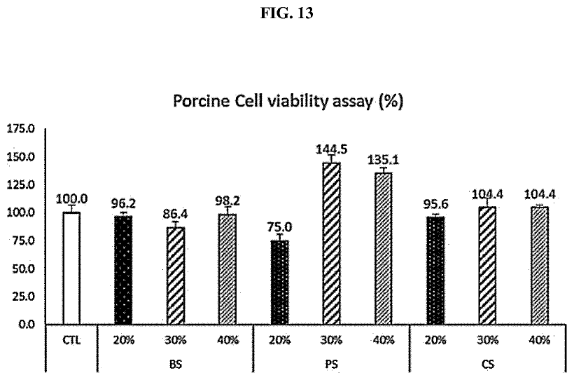

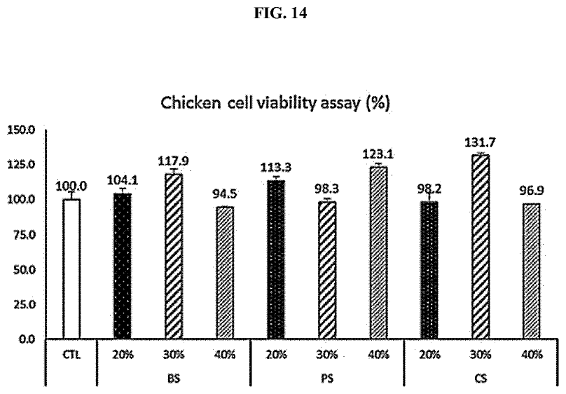

Also, as evidenced in FIG. 10 and FIG. 11, in the case of myoblasts cultured based on chicken cells, the CS treatment group showed the most cell proliferation, and more cell growth was observed than in bovine cells. Particularly, more cell growth was observed in the CS treatment group than in the control group (CTL) with 20% (v/v) FBS added medium. Therefore, it was observed that the cell differentiation in the chicken cells treated with CS was similar to CTL, and other treatment groups, although relatively less dense, differentiated into cells without toxicity in a form similar to the control group. Additionally, as in the previous experiment, it was observed that cell growth was more predominant in serum (BS and CS) than plasma (BP and CP), and in chicken blood than in bovine blood.

This result indicates that using chicken blood (CP and CS) instead of the control group (CTL) FBS can significantly increase the growth and proliferation of chicken cells.

Since the blood obtained at the age of livestock butchering was used, such a trend was not distinctly observed in bovine cells. As evidence, the control group of bovine cell experiments using a medium added with fetal bovine serum showed the most excellent cell growth and proliferation.

### 3-3. Verification of the Promoting Effect on Myoblast Growth and Cell Toxicity Using the FBS Substitute of the Present Invention Using the FBS Substitute as a Complete (100%) Substitute in the Growth Medium

The effects on myoblast differentiation and proliferation were verified using each FBS substitute (bovine serum (BS), porcine serum (PS), and chicken serum (CS)) produced by the method of Example 1 from the blood of three representative livestock species (bovine, porcine, chicken). Satellite cells obtained in Example 2 from each species were used, and cells were cultured in the same manner as in Example 3-2, except that the FBS in the cell growth and proliferation medium was completely substituted (100% substituted). The MTT assay was performed using the same measurement method as described in Example 3-1.

As shown in FIG. 12, in bovine cells, when BS and PS were added at the same amount as FBS, 20% (v/v), it was found that they could sufficiently substitute FBS. In contrast, CS showed about 70% cell survival rate when substituted up to 30% (v/v), and adding more resulted in negative effects.

As shown in FIG. 13, in porcine cells, when BS or CS was added at 20% (v/v), the cells survived to an almost similar extent as FBS. Adding more than that was not effectively proportional to the increase in cell survival rate. In the case of PS, adding 30% (v/v) showed about 1.4 times higher effect than FBS.

As shown in FIG. 14, in chicken cells, all treatment groups showed similar or slightly higher cell survival rates than FBS when added at 20% (v/v). In the case of BS and CS, increasing the addition amount to 30% (v/v) could slightly enhance the cell survival rate.

The above description of the present invention is for illustrative purposes, and those skilled in the art of the present invention will understand that the present invention can be easily modified into other specific forms without changing the technical spirit or essential features of the invention. Therefore, the embodiments described above should be understood as exemplary in all respects and not limiting.

### INDUSTRIAL APPLICABILITY

Using the cultured meat medium additive (FBS substitute) produced by the unique method of the present invention, it is possible to completely substitute or use in combination with the fetal bovine serum (FBS), which has high conventional costs and ethical issues of extraction from living calves, thereby reducing the amount of FBS used and improving the production efficiency of cultured meat. Furthermore, it enhances the utilization and value of butchery by-products, which are necessarily generated during the butchering of livestock for meat production, leading to cost savings in disposal, increased income, and other economic benefits, making it highly industrially applicable.

1. A method for preparing a medium additive for culturing cultured meat from butchery by-products, comprising:
(i) a step of obtaining serum or plasma from blood separated from animals other than humans as a by-product of butchering;
(ii) a step of bleaching the obtained serum or plasma from the step (i); and
(iii) a step of filtering the bleached serum or plasma obtained from the step (ii).

2. The method of claim 1, wherein the animal is one or more selected from the group consisting of cattle, pigs, and chickens.

3. The method of claim 2, wherein the cattle are 30 to 48 months of age, the chickens are 2 to 8 weeks of age, and the pigs are 180 days of age.

4. The method of claim 1, wherein the bleaching comprises mixing kaolin with the serum or plasma obtained in the step (i).

5. The method of claim 1, wherein the bleaching is performed at a temperature of 50 to 70° C.

6. The method of claim 1, wherein the filtering comprises filtering through pores sized 0.05 to 0.3 μm in diameter.

7. The method of claim 6, wherein the filtering further comprises a pre-filtering process through pores sized 2 μm to 25 μm in diameter.

8. The method of claim 6, wherein the filtering comprises using a membrane filter.

9. The method of claim 8, wherein the filtering further comprises using a method selected from the group consisting of filter paper, syringe filter, and combinations thereof.

10. The method of claim 1, wherein the method further comprises sterilization process after the filtering step of (iii).

11. The method of claim 10, wherein the sterilization is performed at a temperature of 50 to 70° C.

12-21. (canceled)

22. A method for preparing cultured meat, comprising a step of proliferating muscle satellite cells using medium compositions for culturing cultured meat.

23. The method of claim 22, wherein the medium composition is for the proliferation of muscle satellite cells.

24. The method of claim 22, wherein the medium composition comprises, relative to 100 weight parts of serum-free medium:
1 to 20 weight parts of the medium additive for culturing cultured meat produced by the method of claim 1; and
0.5 to 1.5 weight parts of antibiotics/antimicrobials, and
wherein the medium additive is for the proliferation of muscle satellite cells.

25. The method of claim 24, wherein the medium additive substitutes for or is used in conjunction with fetal bovine serum (FBS).

26. The method of claim 24, wherein the serum-free medium is at least one selected from the group consisting of Ham's 10 medium, Ham's 12 medium, Dulbecco's Modified Eagle Medium (DMEM), Minimum Essential Medium (MEM), MCDB-201 medium, and RPMI 1640 medium.

27. The method of claim 24, wherein the antibiotics/antimicrobials are at least one type of antibiotic selected from penicillin and streptomycin; and
at least one type of antifungal agent selected from amphotericin-B, clotrimazole, miconazole, ketoconazole, and nystatin.

28. The method of claim 22, wherein the medium composition additionally comprises fetal bovine serum (FBS).

29. The method of claim 28, wherein the medium composition comprises, relative to 100 weight parts of serum-free medium:
10 to 20 weight parts of the medium additive for culturing cultured meat produced by the method of claim 1;
0 to 20 weight parts of fetal bovine serum (FBS); and
0.5 to 1.5 weight parts of antibiotics/antimicrobials.
